(19) United States
(12) Patent Application Publication
(10) Pub. No.: US 2009/0043218 A1
(43) Pub. Date: Feb. 12, 2009

(54) Tachyarrhythmia detection, differentiation and assessment

(76) Inventors: Robert A. Warner, Tigard, OR (US); Patricia A. Arand, McMinnville, OR (US); Peter T. Bauer, West Linn, OR (US)

Correspondence Address: ROBERT D. VARITZ, P.C., 4915 SE 33RD PLACE, PORTLAND, OR, 97202, US

(21) Appl. No.: 12/221,845

(22) Filed: Aug. 6, 2008

Related U.S. Application Data
(60) Provisional application No. 60/963,978, filed on Aug. 7, 2007.

(51) Int. Cl. A61B 5/0464; A61B 5/046

(52) U.S. Cl. 600/516; 600/518

(57) ABSTRACT

A method employable during a tachycardia-tachyarrhythmia condition in a person for detecting, verifying and distinguishing ventricular and supra-ventricular tachyarrhythmias, including ventricular fibrillation, including (a) confirming the presence of a tachyarrhythmia heart rate, (b) on such confirmation, collecting time-frame-simultaneous ECG and heart-sound information, (c) following such collecting, choosing selected ECG time-span, and heart-sound intensity, data, and (d) utilizing the chosen, selected ECG time-span, and heart-sound intensity, data, characterizing the defined condition as resulting from one of (a) supra-ventricular tachyarrhythmia, (b) ventricular tachyarrhythmia, and (c) ventricular fibrillation.

### CROSS REFERENCE TO RELATED APPLICATION

This application claims priority to currently co-pending U.S. Provisional Patent Application Ser. No. 60/963,978, filed Aug. 22, 2007, for “Tachyarrhythmia Detection, Assessment and Differentiation Utilizing Common Time Base ECG and Heart Sound Information”. The entire disclosure content of that prior-filed provisional application is hereby incorporated herein by reference.

### BACKGROUND AND SUMMARY OF THE INVENTION

This invention relates to methodology for the detection, differentiation, assessment and characterization of tachyarrhythmias (ventricular, including ventricular fibrillation, and supra-ventricular—all of which are conditions within the term “tachycardia”), and very specifically to such methodology which is based upon computer analysis of gathered, common-time-base, ECG and heart sound information. The invention rests, in part, upon the recognition that, by acquiring, and suitably thereafter computer-examining and combinedly processing, certain, special categories of such gathered information, it is possible, and even relatively simple, very accurately to detect and differentiate, particularly, ventricular and supra-ventricular tachyarrhythmias for critical assessment purposes. Detection and categorization also of ventricular fibrillation lies within the practice of the invention.

For a variety of clinically important reasons, and in the context of examining a person's heart condition, it is necessary, and indeed critical, to be able to perform accurate detections, differentiations and diagnoses of tachyarrhythmias of the very different types just mentioned above. In this field of heart-activity investigation, we treat, for illustration purposes herein, a heart-beat rate above about 100-beats-per-minute as a tachycardia condition, also referred to herein as a tachycardia-tachyarrhythmia condition extant in a person. Elaborating a bit something which was just mentioned, a tachyarrhythmia condition which is of interest in the setting of the present invention, within the realm of tachycardia, is the mentioned supra-ventricular tachyarrhythmia event. Such an event includes two kinds, one of which involves a tachyarrhythmia condition accompanied by some form of an abnormal, additional characteristic created by a pathological condition, such as a Bundle Branch Block (BBB) conduction defect, and the other of which is not so accompanied.

Of the several mentioned tachyarrhythmia kinds (four in number), and short of ventricular fibrillation, ventricular tachyarrhythmia is the most dangerous one, inasmuch as it can rapidly deteriorate to ventricular fibrillation—a deterioration to an extremely dangerous condition which can quickly become a life-threatening, terminal event.

The earlier that a ventricular tachyarrhythmia event is detected, the easier it is to convert such an event to sinus rhythm. Critically, confusion of an apparent ventricular fibrillation with either ventricular tachyarrhythmia or supra-ventricular tachyarrhythmia can result in the attempted application of a dangerously inappropriate, but intended-to-be-corrective countershock, i.e., a synchronous, as distinguished from an asynchronous, D. C. countershock.

Importantly, accurate identifications of truly non-lethal episodes of tachyarrhythmia can, using implanted defibrillators and/or appropriate antiarrhythmic drugs, lead to effective interventions to prevent future lethal episodes. Errors of detection/identification made either by humans or by a computer algorithm can cause the inappropriate administration, or the withholding, of counter shocks or effective, but potentially dangerous drugs.

Current methods of characterizing tachyarrhythmias typically use only ECG criteria, and are imperfect for reasons, such as:

1. QRS morphologies associated with supra-ventricular tachyarrhythmia associated with aberrant conduction, and ventricular tachyarrhythmia, are often similar in many respects;

2. The most definitive way to diagnose re-entrant ventricular tachyarrhythmia as the cause of a wide, complex tachyarrhythmia is to demonstrate AV (atrioventricular) dissociation. However, especially in the presence of tachyarrhythmia, the P waves needed to establish the presence of AV dissociation are often difficult to discern within the overall ECG complex.

According to the present invention, a methodology is proposed for detecting, discriminating and enabling accurate, diagnostic assessments of tachyarrhythmias of the types mentioned above utilizing selected information in categories drawn from both gathered ECG, and gathered heart-sound, data acquired from a person. We have discovered, importantly, the fact that the use of collected and suitably computer-processed heart sounds, along with common-time-base, computer-processed ECG data, can significantly improve the accuracy/reliability of information leading to correct diagnoses of the mentioned types of tachyarrhythmias.

A number of comments and factors are notable here with regard to understanding how practice of the present invention, and the thinking behind it, both based upon relatively simple and elegant ECG and heart-sound data gathering and computer algorithmic processing, can lead to significant tachyarrhythmia differentiation and diagnostic accuracy.

In the context of focusing upon data which is relevant to performing an accurate tachyarrhythmia detection and differentiation, we have come to recognize that the use of gathered and observed heart sounds, along with relevant ECG information, can significantly improve the diagnostic accuracy relating to the several kinds of tachyarrhythmias with respect to which the methodology of the present invention is concerned.

For example, compared to supra-ventricular rhythms which take place without aberrant intra-ventricular conduction, bundle branch block (BBB) and ventricular tachyarrhythmia are associated with slow, initial ventricular activation because (a) the initial activation activity takes place through ventricular muscle that has about 20% of the conduction velocity of Purkinje tissue, and (b) such initial activation occurs via a more circuitous pathway than does supra-ventricular activation. Consequently, there is an abnormally long QRS interval associated with BBB and ventricular rhythm. The QRS interval often, but not always, longer in the latter, than in the former, case.

By definition, AV dissociation exhibits a continually changing ECG PR interval. Since, other things being equal, the loudness of the so-called S1 heart sound is inversely related to the duration of the mentioned PR interval, therefore, in AV dissociation, there are beat-to-beat changes in the intensity of the S1 heart sound.

At what might be thought of as the extreme end of the tachyarrhythmia spectrum, namely, in a ventricular fibrillation situation, the various heart sounds are either greatly reduced in intensity, or are completely absent. This observation can definitively confirm the presence of ventricular fibrillation, and can distinguish ventricular fibrillation from a similarly looking electrical condition, e.g. as would be created by a loose electrical ECG lead.

Using the combination of ECG and cardiac acoustical data to diagnose tachyarrhythmias requires accurate timing of heart sounds with respect to simultaneously acquired ECG information. Here, identifying the onset of the QRS interval in an ECG waveform is especially important. In many tachyarrhythmia situations, there is an undulating baseline, or a gradual or irregular slope of the initial portion of the QRS interval. This condition makes it difficult to establish QRS onset as an appropriate fiducial point using criteria that only involve hunting for an abrupt, “lead-in” departure of the QRS portion of an ECG waveform from baseline. In this setting, what will be briefly described herein as a “zero crossing” method provides an excellent, alternative way algorithmically to analyze the shapes of different portions of a waveform in order to obtain fiducial-point data. This method employs a series of lines above, below, and in all circumstances parallel to, the normal baseline. The method effectively then records and analyzes the times at which the erratic waveform of interest crosses each of the parallel lines, and also analyzes the timing relationships between ECG zero crossings and the time locations of the several heart sounds.

Another thought which is discussed below and illustrated in relation to practicing the methodology of the present invention is that, in certain instances, it may be very useful to employ conventionally calculated statistical Z-scores (i.e., “standard” scores), instead of absolute values, such scores being derived from various, observed parameters, in order to achieve certain, immediately intuitively understandable data-presentation advantages. For example, such scores permit conclusions about the statistical significances of differences from “normal” or from “baseline”. Z-scores readily account for the inevitable variability in acquired data, whether such variability is due to biological differences, or to errors in measurement. Additionally, Z-scores express all associated data on the same scale—a very useful consideration, particularly where multi-parameter monitoring is involved. Further regarding the use of Z-scores, implications of similar numerical differences in acquired values become uniform throughout the entire distribution of values. This uniformity makes it easy for users to comprehend and employ the output information derived from practice of the invention.

The use of such Z scores in the context of implementation of the present invention may also be associated with the highly effective, companion use of predetermined colors that are linked to ranges of Z-scores. Such a color linkage furnishes a powerful, tool for conveying immediate, “importance-characterization”, intuitive information regarding tachyarrhythmia characterization and assessment output results.

Touching here on certain terminology which is employed in the description and characterization of the present invention, the concepts of “normal” and “normalcy”, spoken of herein variously, are applied in a “measurement manner” to certain important ECG and heart-sound parameters, including QRS interval, S1 heart-sound intensity, S1 heart-sound intensity variation from heart-beat-to-heart-beat, and QS1 interval. In this context, normal, or normalcy, as measurement “markers” is/are based upon either (a) person-specific, normal, heart-functionality baseline data available and acquired from a person under circumstances not affected by any appreciable heart-function problem, or (b) an available, general database of such information relating to, and drawn from, a population of people (“normals”) having physical, etc. characteristics appropriately related to those of a subject person whose heart activity is being then observed.

Speaking here somewhat more specifically about the important S1 heart-sound data aspect of the present invention, the intensity of the S1 heart sound can be used as a parameter for distinguishing supra-ventricular from ventricular tachyarrhythmia. Supra-ventricular tachyarrhythmia is often associated with increased forcefulness of left ventricular contraction, partly because of the “force-frequency” relationship associated with tachyarrhythmia, per se. Increased sympathetic activity associated with supra-ventricular tachyarrhythmia can also contribute to stronger ventricular contraction. The increased forcefulness of left ventricular contraction imparts more kinetic energy to mitral leaflet closure and therefore increases the S1 heart-sound intensity. Conversely, marked ventricular dyssynchrony associated with ventricular tachyarrhythmia reduces the forcefulness of left ventricular contraction and therefore also decreases the S1 heart-sound intensity. In the event that ventricular tachyarrhythmia degenerates to ventricular fibrillation, mechanically effective left ventricular contraction ceases, and the S1 heart-sound intensity diminishes to zero.

Continuing with background reference still to the S1 heart-sound intensity issue, and more specifically to S1 heart-sound intensity variability, this tachyarrhythmia-assessment indicator is calculated by dividing the standard deviation of acquired S1 heart-sound intensity measurements by the mean of these measurements during the period of acquisition-recording. S1 heart-sound intensity and variability during a tachyarrhythmia being evaluated are considered abnormal if their respective means differ statistically significantly from the patient's own baseline data, mentioned above, or from the data drawn from a population of known “normals”, also mentioned above

According to the present invention, and specifically to the core methodology of this invention, relevant and important information about tachyarrhythmias is obtainable substantially solely (a) by observing heart rate, per se, in order to determine confidently that a potential tachyarrhythmia event is underway, (b) by collecting ECG data, and choosing, from that collected data selected time-span data (QRS interval), and (c) by additionally and collaboratively collecting and employing heart-sound (S1 heart sound) intensity data, per se, as well as data regarding variability in such heart-sound intensity from the heart-beat-to-heart-beat. With such collected data available, another important tachyarrhythmia-assessment data interval is obtained which interrelates heart electrical behavior and heart acoustic behavior—the QS1 time interval.

In a somewhat specific sense, the present invention may be expressed as a method for detecting, verifying and distinguishing ventricular and supra-ventricular tachyarrhythmias, and also ventricular fibrillation, during a tachycardia-tachyarrhythmia event including the steps of (a) confirming the presence of a tachyarrhythmia condition in a person, and on such a confirmation, (b) collecting time-frame-simultaneous ECG and heart-sound information, (c) from the collected ECG information, determining whether the time duration of the QRS complex (interval) in each heartbeat is different from (and in a more particular sense longer than) normal, (d) from the collected heart-sound information, determining whether selected, respective heart-sound intensities (preferably focused on the S1 heart sound) are at least one of (1) different from normal, and (2) notably irregular from heartbeat-to-heartbeat, and (e) utilizing collectively the results of the mentioned, determining steps, appropriately characterizing the tachyarrhythmia condition as being one of (a) supra-ventricular tachyarrhythmia, (b) ventricular tachyarrhythmia, and (c) ventricular fibrillation.

Output results—differentiations, characterizations, and assessments—derived from computer algorithmic processing applied to the collected data may be furnished in terms of statistical Z-scores, and these scores may preferably, when employed, be accompanied by intuitively associated Z-score-range-associated colors.

The various features and advantages which are offered by the methodology of the present invention will now become more fully apparent as the description which follows in detail below is read in conjunction with the accompanying drawings.

### DESCRIPTIONS OF THE DRAWINGS

FIG. 1 is a block/schematic diagram generally illustrating the overall methodology of the present invention in its preferred and best-mode form.

FIGS. 2, 3 and 4 provide graphical and stylized illustrations of ECG time-span, and heart-sound intensity, data characteristics which are relevant to the practices of tachyarrhythmia detection, differentiation and assessment in accordance with implementation of the invention as generally outlined in FIG. 1.

FIG. 5 provides a time-based graphical illustration of utilization of a so-called a zero-crossing practice employable in certain instances in relation to implementation of the steps of the invention.

FIGS. 6 and 7 are block/schematic diagrams illustrating, respectively, two different manners of practicing the invention, as such is generally illustrated in FIG. 1, with specific reference to the calculation and assessment-use of so-called Z-scores linked with the presentation of score-associated, intuitive colors.

FIG. 8 furnishes, in a chart and grid form, one illustration of a tachyarrhythmia differentiation and assessment matrix relevant to the practice of the invention as illustrated in FIGS. 1-4, inclusive, and 6 and 7.

FIG. 9 furnishes an illustration of the calculations and uses of Z scores and associated intuitive colors in relation to the practices of the methodology of the present invention pictured in FIGS. 6 and 7.

FIG. 10 furnishes, in a chart and grid form, another illustration of a tachyarrhythmia differentiation and assessment matrix relevant to the practice of the invention.

### DETAILED DESCRIPTION OF THE INVENTION

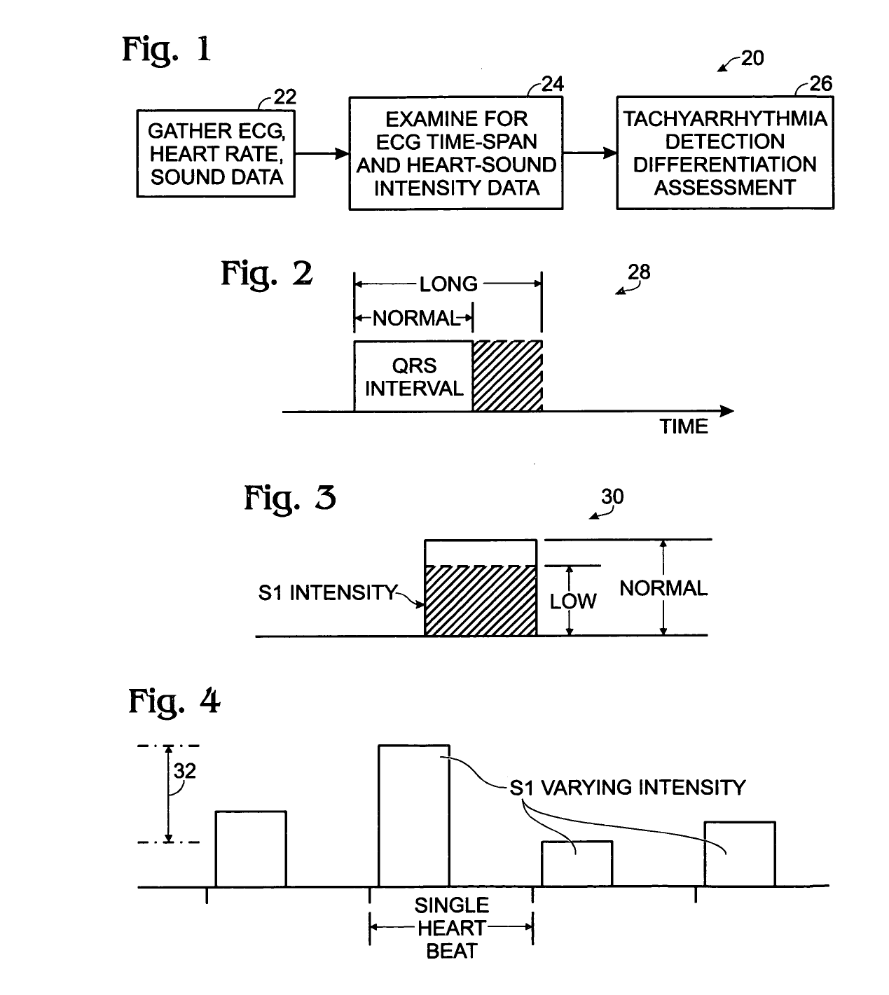

Turning now to the drawings, and referring first of all to FIGS. 1-4, inclusive, indicated generally in block/schematic form at 20 in FIG. 1 is the structure of the methodology of the present invention for tachycardia/tachyarrhythmia detection, differentiation, characterization and assessment. Methodology 20 is illustrated specifically in FIG. 1 by three labeled blocks 22, 24, 26.

Block 22 represents the step, or steps, involved in gathering, or collecting, time-frame-simultaneous ECG and heart-sound information, along with important heart-rate information. Heart-rate is obtained herein from measurements of the RR interval to which the heart rate is inversely related. This heart-rate information is needed, of course, for identifying and confirming, basically, whether or not a potential tachyarrhythmia condition which is to be detected and characterized by the present invention is in fact underway with respect to a particular person. This condition is referred to herein as a defined tachycardia-tachyarrhythmia condition extant in a person. To this end, and for the purpose of description of the preferred and best-mode manner of practicing the invention as illustrated herein, we define, as a “marker” of a probable tachycardia-tachyarrhythmia condition of interest, a condition which is characterized by a heart-beat rate essentially greater than about 100-beats-per-minute (mentioned above)—an RR interval of less than about 0.6-seconds.

Accordingly, in terms of the practice of the present invention now to be described, if the steps performed by block 22 do not detect a heart rate above this “marker” level, no further inquiry is necessary. Having said this, we do note that practice of the present invention is not constrained simply to events wherein a detected heart rate exceeds about 100-beats-per-minute, recognizing that those skilled in the art wishing to employ various features of the methodology of the present invention might well choose another, suitable “marker” heart-beat number.

It is especially important to note that, in addition to gathering heart-beat-rate information, block 22 functions importantly to gather both ECG time-span information, shortly to be more fully described, and additionally, heart-sound intensity information, particularly focused on the intensity level of the so-called S1 heart sound, and on certain intensity-level variations in this sound.

The information specifically gathered regarding ECG time-span and S1 heart-sound intensity level, which information might typically be collected over a suitable number of heart beats, or cardiac cycles, such as about 100- to about 200-cycles, is, within block 24, then processed in the environment of a suitably, algorithmically programmed digital computer to generate certain, very specific information, as follows.

In accordance with practice of the present invention, the ECG time-span information which is important is especially the time duration of the QRS interval. Specifically, what is important to determine is whether this interval has a normal length of about up to, but not beyond, about 100-milliseconds. This time-interval determination, performed conventionally by block 24, is illustrated very generally and schematically at 28 in FIG. 2. How is this QRS time interval information is used in terms of tachyarrhythmia characterization will be explained shortly. The shaded area in FIG. 2 represents notable lengthening of the QRS time interval beyond “normal”.

S1 heart-sound intensity information is examined by conventional techniques in block 24 from two different points of view. In the first, a determination is made regarding whether, in general terms, the S1 heart-sound intensity is higher or lower than a normal intensity. This is practice illustrated generally and schematically at 30 in FIG. 3 where the shaded area here highlights this consideration.

In the second S1 heart-sound intensity view point, a determination is made to assess whether, from heart-beat to heartbeat, the S1 heart-sound intensity is appreciably variable. Such a noted variation is indicated generally by a double-headed arrow 32 in FIG. 4.

Finally, block 24 determines the time nature of the QS1 interval, a practice which further uniquely links the worlds of heart electrical activity and heart acoustic activity in the process of the invention for differentiating, characterizing and assessing the various mentioned tachyarrhythmias. Notable, in this regard are QS1 intervals which are larger than normal.

In relation to tachyarrhythmia-assessment practice in accordance with the present invention, certain, different, respective, data-parameter patterns of (a) QRS time interval notably longer than normal, (b) S1 heart-sound intensity notably lower or higher than normal, (c) notably greater-than-normal heartbeat-to-heartbeat variations in this intensity, and (d) QS1 time interval notably larger than normal, each lead, as will now be explained, effectively to clear, respective identifications, differentiations and characterizations of the several different kinds of tachyarrhythmia events whose presences are the focus of the methodology of the present invention.

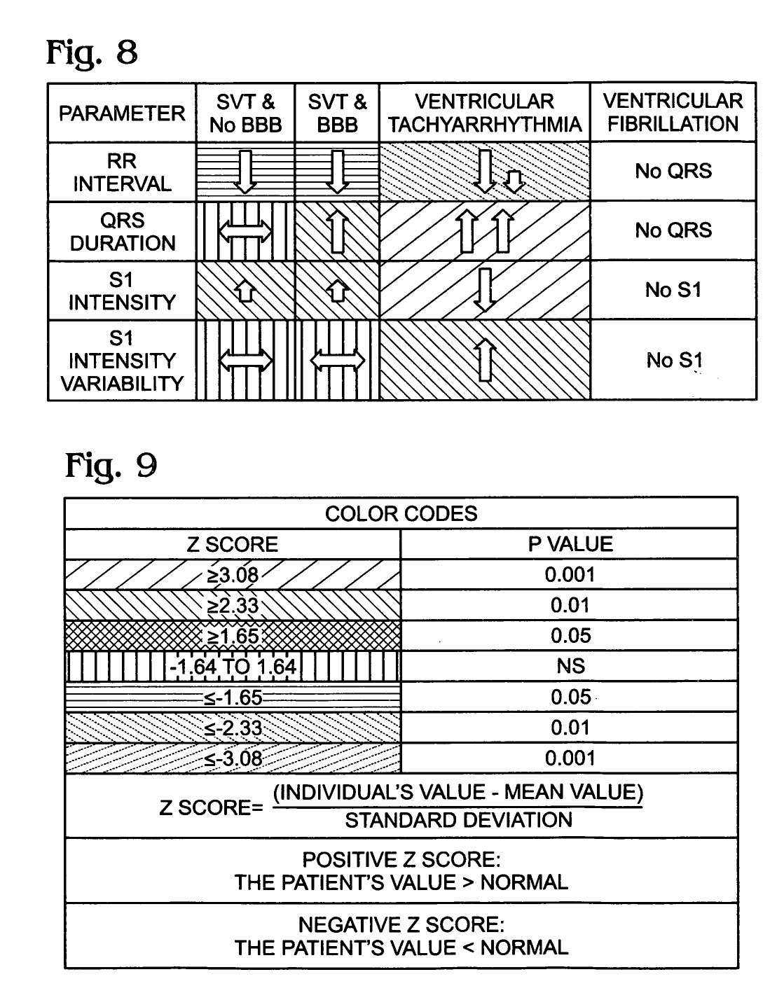

Turning attention now to FIG. 8, in relation to the block/schematic diagram of FIG. 1, and particularly initially just on certain aspects of FIG. 8, seen here, in a grid-like, chart form, is a tachyarrhythmia distinguishing, characterizing and assessing matrix which illustrates how certain ones of the ECG and S1 heart-sound information-derived and calculated parameters mentioned above lead to the desired tachyarrhythmia characterizations, etc. The matrix of FIG. 8, and specifically a particular one (normally only one) of the assessments presented in this figure, will be created, and presented as output, by block 26 in FIG. 1 in relation to the detection of an tachyarrhythmia condition extant in a given person.

As can be seen, presented across the top of the chart pictured in FIG. 8 are the four, different, subject tachyarrhythmia events: (a) SVE & No BBB, referring to supra-ventricular tachyarrhythmia without bundle branch block; (b) SVE & BBB, referring to supra-ventricular tachyarrhythmia accompanied by bundle branch block; (c) Ventricular Tachyarrhythmia; and (d) Ventricular Fibrillation. Progressing downwardly on the left side of the chart in FIG. 8 are listed four of the ECG and S1 heart-sound block-24-determined parameters derived from collected and processed ECG and S1 heart-sound information. These four parameters include heart-rate interval, (labeled RR Interval, which, of course, is related inversely to heart rate, per se), QRS Duration (the earlier herein mentioned QRS time interval), S1 (heart-sound) Intensity, per se, and S1 (heart-sound) Intensity Variability.

Pictured in the rectangles which represent characterization and differentiation assessment intersections of parameters and tachyarrhythmia events in the chart of FIG. 8 are (a) downwardly and upwardly pointing, single-headed arrows, one of the downwardly pointing, and two of the upwardly pointing, arrows being shorter than the others illustrated, (b) three, horizontally disposed, double-headed arrows, and (c) certain text labeling which appears under the event heading Ventricular Fibrillation. The horizontally disposed arrows represent determined parameter values which are normal in nature. The single, downwardly pointing arrows represent parameter values which are lower than normal, with the presentation which includes two, downwardly pointing arrows, one of which is the shorter arrow just mentioned above, indicating an appreciably lower-than-normal value. The single, upwardly pointing arrows indicate generally higher than normal values, with the two presentations each possessing shorter, upwardly pointing arrows indicating slightly greater-than-normal values, and the one presentation having a pair of long, upwardly pointing arrows indicating a value which is significantly higher than normal.

Thus, what one can see from these characterization and differentiation assessment intersections is that supra-ventricular tachyarrhythmia without bundle branch block is distinguished and characterized on the basis that there is an elevated (tachycardia-tachyarrhythmia) heart rate (a shorter-than-normal RR Interval, a QRS Duration having a normal value, an S1 heart-sound Intensity value which is somewhat elevated, and an S1 heart-sound Intensity Variability value (heart-beat to heart-beat) which is normal.

The condition of supra-ventricular tachyarrhythmia accompanied by bundle branch block is distinguished and characterized by a tachycardia-elevated heart rate, by a higher than normal QRS Duration, by S1 a heart-sound Intensity value which is somewhat elevated, and by a normal S1 heart-sound Intensity Variability value.

A ventricular tachyarrhythmia event is characterized by a significantly elevated tachycardia heart rate, a significantly increased QRS Duration, a lower-than-normal S1 heart-sound Intensity, and an elevated S1 heart-sound Intensity Variability.

The most dangerous of all of the tachyarrhythmia events, namely, ventricular fibrillation, is distinguished and characterized by the several conditions clearly set forth in text labeling in the three parameter intersections which exists with the column headed Ventricular Fibrillation in FIG. 8.

The event assessment results presented in FIG. 8 come from the processing and calculations performed in and by block 24, 26 in FIG. 1. Such a results presentation is, of course, an automated presentation which uniquely, and significantly, does not depend upon human, visual interpretation of acquired ECG and heart-sound data.

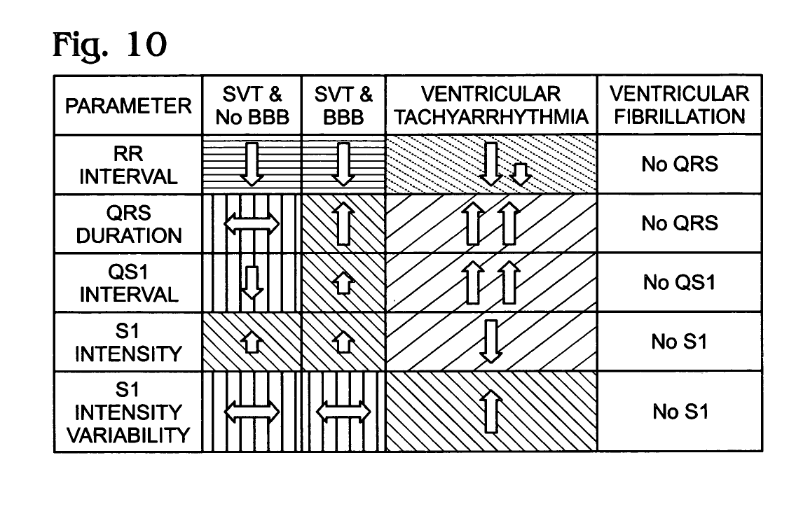

Shifting attention here to FIG. 10, as was mentioned earlier, yet another very useful parameter derived from collected ECG and heart-sound intensity information is the QS1 time interval—an interval which blends important information relating the electrical activity of the heart to the mechanical, acoustic activity thereof. This QS1 interval is the time interval from the Q fiducial point in the ECG waveform essentially to the point in time where the S1 heart sound reaches its maximum intensity.

Whereas the earlier-discussed QRS time interval indirectly suggests information about the strength of ventricular contraction, the QS1 time interval much more directly gives information about the forcefulness of ventricular contraction.

Accordingly, and as can be seen in FIG. 10, a line-item parameter labeled QS1 Interval is here shown added to the previously described assessment, etc. matrix. In this line item, SVT & No BBB is indicated by a slightly decreased QS1 Interval, SVT & BBB is indicated by a slightly enlarged QS1 Interval, Ventricular Tachyarrhythmia is indicated by markedly greater-than-normal QS1 Interval, and Ventricular Fibrillation is indicated by the absence of a QS1 Interval.

The description of the implementation and practice of the present invention provided so far herein has been presented on the basis of an assumption that the QRS and heart-sound waveforms are clearly readable with respect to particular points in time relating to them. There are circumstances involving tachyarrhythmias, however, wherein, for example, with respect to a collected ECG waveform, this waveform appears in such a fashion that it is very difficult to identify the fiducial point known as Q-onset.

FIG. 5 illustrates generally at 33 a relatively well-known zero-crossing signal-processing technique which can be employed under such a circumstance in order to detect with reasonable accuracy, the exact point in time relating to the Q-onset fiducial point. Computer processing implemented within previously described block 24 is brought into play to carry out such a zero-crossing analysis. Generally speaking, this computer processing effectively generates a series of amplitude-offset baselines which substantially parallel one another and also parallel what might be thought of as the nominal baseline—a line which is shown as a thickened, horizontal line 33a in FIG. 5. From this technique, an important point in time, such as the Q-onset point may readily be determined in order to allow for continued signal processing to develop the determined electrical and acoustic parameters which lead to the generation of an assessment matrix such as the matrix set forth in FIG. 8.

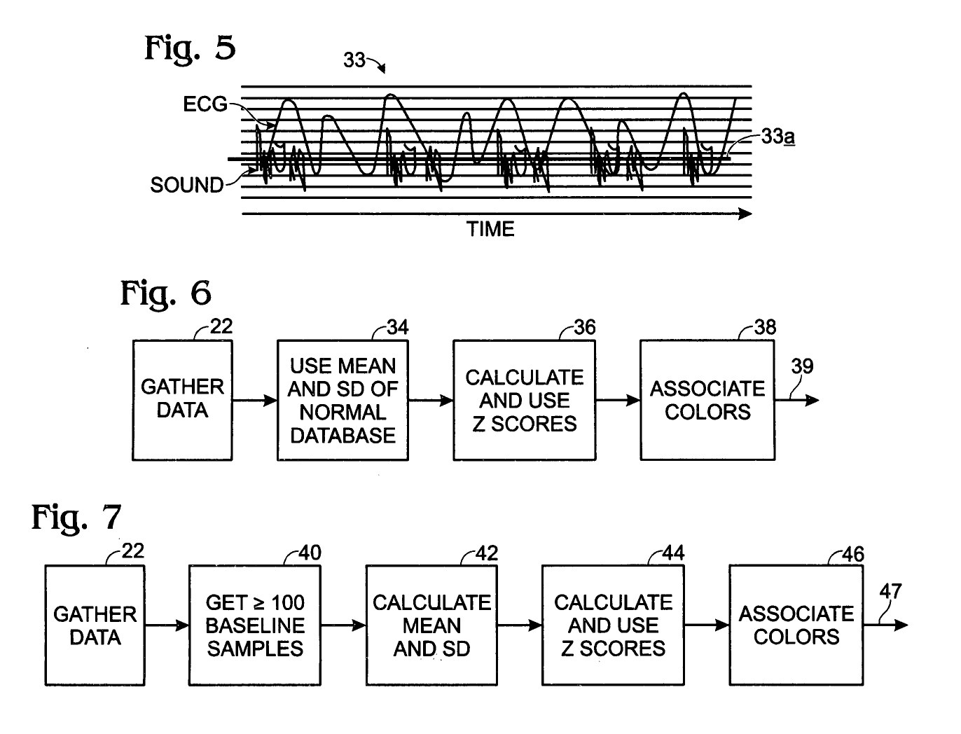

Turning attention now to FIGS. 6 and 7, these two figures illustrate, in more detailed fashions, two, slightly different, methodologic structures for previously described block 24 in FIG. 1. Specifically, these two figures describe two slightly different ways in which statistical Z-scores may be generated (in a conventional fashion) to furnish the characterization and assessment advantages which have been mentioned earlier herein.

Regarding what might be thought of as the Z-score procedure employed in accordance with what is shown in FIG. 6, three blocks numbered 34, 36, 38 illustrate this practice. In FIG. 6, block 22 is essentially the same block 22 shown in, and discussed with respect to, previously described FIG. 1. Blocks 34, 36, 38 represent steps of data processing which are performed in previously described block 24. With respect to block 34, appropriate access is made to a conventional database of ECG and heart-sound information related to clinically normal persons who have demographic characteristics similar to those of a particular person whose condition is currently being investigated. From this database, appropriate mean data and standard-deviation information are derived and are provided to block 36 wherein a conventional Z-score set of calculations is performed. In block 38, predetermined and selected ranges of Z-score values are associated, respectively, with intended intuitive colors that may then be employed within previously mentioned block 26, via communication thereto through processing flow line 39 in FIG. 6, to include such colors appropriately in an assessment matrix, such as in the assessment matrices of FIGS. 8 and 10. Various grey-scale values are seen to appear as background fields in these two matrices to represent the appearances of such Z-score-associated colors.

Referring, in this color-association context, for a moment to FIG. 9 in the drawings, among other things, FIG. 9 illustrates, on its left side, a typical user-chosen, selected range of Z-score values, beneath which, the well-known, conventional calculation approach for determining a Z-score value is clearly presented. In FIG. 9, to the right of the left-hand column in this figure which presents the just-mentioned selected ranges of Z-score values, there is another column labeled P Value, within which are numbers that indicate the probabilities that the differences which exist between normal parameter values, and respective, calculated Z-score values can be attributed to chance alone.

The use of such colors, obviously, provides an extremely intuitive way for an observer to understand quickly calculated characterization, differentiation and assessment results, such as those presented in FIGS. 8 and 10

FIG. 7 is similar to FIG. 6, with the exception that, in this illustrated practice of the invention, Z-scores are calculated on the basis of the ECG and heart-sound data collected prior to the onset of a tachyarrhythmia event (i.e., during a “baseline” period) directly from the particular person whose heart condition is currently being examined to explore a potential tachyarrhythmia event.

The practice of the invention as illustrated in FIG. 7 is set forth in five blocks which are numbered 22, 40, 42, 44, and 46. Block 46 connects effectively with previously discussed block 26 in FIG. 1 via a processing flow line 47 seen in FIG. 7. Blocks 22, 44, 46 are, in the illustration of FIG. 7 essentially the same in structure and performance as blocks 22, 36, 38, respectively, in FIG. 6. In general terms, what takes place within the practices represented by blocks 40, 42, 44, 46 occur within the realm of previously described block 24. Block 40 represents a practice of gathering a large number of ECG and heart-sound data, say for a stretch of time including more than about 100-heart-cycles, in order to develop a baseline sample data from which, in block 42, mean and standard deviation calculations leading to the calculation of Z-scores may take place.

The present invention thus proposes a new and very effective methodology for detecting, characterizing, and assessing the four, important categories of tachyarrhythmia events which have been discussed above in the background, summary and detailed description of the invention. Uniquely, practice of the invention involves the utilization of both collected heart electrical information and heart acoustic information, with the heart electrical information focused principally upon the QRS interval, and the heart acoustic information focused principally on S1 heart-sound intensity and S1 heart-sound intensity variation from the heart-beat-to-heart-beat.

Additional helpful information is obtained by uniquely directly linking electrical information and heart-sound intensity information in a parameter which is based upon both categories of information, namely, the QS1 time interval which is measured between the time of Q-onset to the time of the peak value of S1 heart-sound intensity within the time frame of a single heart beat.

Computer processing based upon the use of a suitably algorithmically programmed digital computer is employed to receive and process collected ECG and heart-sound information, to perform certain calculations regarding that information, and thereafter automatically (i.e., without human intervention, and without requiring human reading of unprocessed data) to generate tachyarrhythmia-condition identifications, assessments and characterizations.

To enhance the intuitive understandability of output results obtained from practice of the invention, collected and processed data is optionally subjected to statistical analysis to generate so-called Z-scores in ranges that define collected-data deviations from so-called normal heart activity, with the added concept that these ranges may be identified by suitably linked and associated, user-selected, intuitive colors which help a reviewer of tachyarrhythmia-assessment results quickly to understand the natures of these results.

Accordingly, while a preferred manner of practicing the invention, in its best currently known mode, and certain modest variations, have been illustrated and described herein, we appreciate that variations and modifications beyond those suggested herein may be made without departing from the spirit of the invention.

We claim:

1. A method employable during a defined tachycardia-tachyarrhythmia condition extant in a person for detecting, verifying and distinguishing ventricular and supra-ventricular tachyarrhythmias, including ventricular fibrillation, comprising
obtaining, from the person, heart-rate information to confirm the presence of such a defined condition,
on such confirmation, collecting, from the person, time-frame-simultaneous ECG and heart-sound information,
following said collecting, choosing selected ECG time-span, and heart-sound intensity, data, and
utilizing the chosen, selected ECG time-span, and heart-sound intensity, data, characterizing the defined condition as resulting from one of (a) supra-ventricular tachyarrhythmia, (b) ventricular tachyarrhythmia, and (c) ventricular fibrillation.

2. The method of claim 1, wherein said choosing of selected ECG time-span data includes choosing QRS interval data.

3. The method of claim 1, wherein said choosing of selected heart-sound intensity data includes choosing (a) S1 heart-sound, per-occurrence, intensity data and (b) S1 heart-sound, heart-beat-to-heart-beat, intensity-variation data.

4. The method of claim 3, wherein said choosing of selected ECG time-span data includes choosing QRS interval data.

5. The method of claim 4, wherein said choosing further includes identifying QS1 interval data, and said characterizing takes into account also such QS1 interval data.

6. The method of claim 5, wherein said characterizing is performed automatically by a computer.

7. The method of claim 5, wherein said characterizing involves utilizing statistical Z-scores in relation to the QRS interval data, the S1 heart-sound intensity data, the S1 heart-sound intensity-variation data, and the QS1 interval data.

8. The method of claim 1, wherein said choosing further includes identifying QS1 interval data, and said characterizing takes into account also such QS1 interval data.

9. The method of claim 8, wherein said characterizing involves utilizing statistical Z-scores in relation to the ECG time-span data, the heart-sound intensity data, and the QS1 interval data.

10. A method employable during a defined tachycardia-tachyarrhythmia condition extant in a person for detecting, verifying and distinguishing ventricular and supra-ventricular tachyarrhythmias, including ventricular fibrillation, comprising
collecting, from the person, time-frame-simultaneous ECG and heart-sound information,
from the collected ECG information, determining whether the QRS interval in each heartbeat is different from normal,
from the collected heart-sound information, determining whether the S1 heart-sound intensities are at least one of (a) different from normal, and (b) irregular from heartbeat-to-heartbeat,
from the combined, collected ECG and heart-sound information, determining whether the QS1 interval in each heart beat is different from normal, and
utilizing combinedly the results of said three determining steps, characterizing the defined condition as resulting from one of (a) supra-ventricular tachyarrhythmia, (b) ventricular tachyarrhythmia, and (c) ventricular fibrillation.

11. The method of claim 10, wherein said determining, insofar as normalcy of the QRS interval is concerned, involves assessing whether the QRS interval in each heartbeat is longer than normal.

12. The method of claim 10, wherein said determining, insofar as normalcy of the S1 heart-sound intensity is concerned, involves assessing whether S1 heart-sound intensities are lower than normal.

13. The method of claim 10, wherein said determining, insofar as normalcy of the QS1 interval is concerned, involves assessing whether the QS1 interval in each heart beat is longer than normal.
